(19) United States
(12) Patent Application Publication
(10) Pub. No.: US 2026/0318861 A1
(43) Pub. Date: Oct. 15, 2026

(54) ELECTROCHEMICAL BIOSENSOR FOR LE

(71) Applicant: CLEU DIAGNOSTICS, LLC, Ardmore, PA (US)

(72) Inventors: Ron H. BIHOVSKY, Wynnewood, PA (US); Ewen Ogilvie BLAIR, Livingston, Scotland (GB); Christopher GEDDES, Livingston, Scotland (GB); Eirini STAMKOU, Livingston, Scotland (GB); David Donogh FITZGERALD, Livingston, Scotland (GB); Christopher Stewart MCMURRAN, Livingston, Scotland (GB); Jeffrey MIRZA, Ardmore, PA (US); Andrew FLEISCHMAN, Ardmore, PA (US)

(21) Appl. No.: 19/432,827

(22) Filed: Dec. 24, 2025

Related U.S. Application Data
(63) Continuation of application No. PCT/US24/35630, filed on Jun. 26, 2024.
(60) Provisional application No. 63/523,213, filed on Jun. 26, 2023.

(51) Int. Cl. G01N 27/327; G01N 33/487

(52) U.S. Cl. CPC G01N 27/3272; G01N 33/487

(57) ABSTRACT

A reagent formulation comprising: (a) an ester-containing substrate with an electrochemical mediator; (b) a buffer having a pH sufficiently low to preserve the substrate prior to introduction of a sample having a higher pH; (c) one or more chemical stabilizers; and (d) one or more surfactants.

### REFERENCE TO RELATED APPLICATIONS

This application claims priority to U.S. Provisional Patent Application Ser. No. 63/523,213, filed Jun. 26, 2023, which is hereby incorporated by reference in its entirety.

### FIELD OF INVENTION

The present disclosure relates generally to electrochemical biosensors and methods of use thereof for the detection of leukocyte esterase (LE) in biological fluids, and, more particularly, for the detection of LE in urine and synovial samples from a patient.

### BACKGROUND

The presence of an abnormally high number of leukocytes (white blood cells [WBCs]) in biological fluid is commonly used as an indicator for the presence of an infection. For example, an elevated number of leukocytes in urine can indicate the presence of a urinary tract infection (UTI). Historically, technicians relied on manually counting leukocytes in urine under direct visualization with a microscope. However, this technique has largely been replaced by chemical detection of LE, an enzyme released by activated leukocytes, which can be used as a proxy for leukocytosis. Chemical detection of LE is simple and is not impacted by WBC lysis, which occurs spontaneously over time after sample collection. A commercial urine dipstick test for LE is now commonly used for the diagnosis of UTI. LE has also been shown to be a surrogate for infection in other biological fluids, such as synovial fluid, cerebrospinal fluid, and peritoneal fluid. Most particularly, there is growing evidence for the utility of using LE as a biomarker for the diagnosis of periprosthetic joint infection (PJI).

Commonly used LE dipstick tests utilize a chromogenic substrate that reacts with diazonium salt to produce a purple azo pigment. The test is then interpreted either optically by an automated instrument or visually by a clinician based on the change in color of the chemical reagent pad. However, Applicant has identified a number of disadvantages to colorimetric tests, in particular when a biological fluid sample being tested is not clear. For example, the color of urine typically ranges from yellow to amber but can also vary significantly with a large range of colors depending on diet, medications, and other substances filtered into urine by the kidneys. Synovial fluid is commonly blood-tinged and turbid after aspiration from a joint cavity. It is common practice to centrifuge a synovial fluid sample before testing with a LE dipstick to reduce the impact of blood or turbidity on the interpretation of the test. However, these factors reduce the reliability of colorimetric LE tests.

Applicant has also identified the benefits of using instead an electrochemical biosensor to test for LE. Electrochemical biosensors can provide precise and sensitive qualitative or quantitative measurements without being impacted by the color of the test sample. However, electrochemical biosensors have their own unique challenges, which must be overcome to produce in vitro diagnostics tests that can be used commercially with an acceptable performance and shelf life.

Therefore, Applicant has identified a need to overcome the challenges posed by electrochemical biosensors in testing for LE in biological fluids. The present invention fulfills this need, among others.

### SUMMARY OF INVENTION

The following presents a simplified summary of the invention in order to provide a basic understanding of some aspects of the invention. This summary is not an extensive overview of the invention. It is not intended to identify key/critical elements of the invention or to delineate the scope of the invention. Its sole purpose is to present some concepts of the invention in a simplified form as a prelude to the more detailed description that is presented later.

One aspect of the present invention is a reagent formulation for a biosensor having a buffer with a low pH. In one embedment, the reagent formulation comprises: (a) an ester-containing substrate with an electrochemical mediator; (b) a buffer having a pH sufficiently low to preserve the substrate prior to introduction of a sample having a higher pH; (c) one or more chemical stabilizers; and (d) one or more surfactants.

Another aspect of the present invention is a reagent formulation for a biosensor having an abundance of surfactant. In one embodiment, the reagent formulation comprises: (a) an ester-containing substrate with an electrochemical mediator; (b) a buffer; (c) one or more chemical stabilizers; and (d) one or more surfactants, at least one being non-ionic, wherein the one or more surfactants is at least 0.1% w/v or 0.05% and 0.2% of the reagent formulation.

Yet another aspect of the present invention is a biosensor in which the reagent does not contact the electrode prior to introduction of the sample. In one embodiment, the biosensor comprises (a) at least one working electrode on a surface; (b) a reagent formulation comprising at least an ester-containing substrate with an electrochemical mediator, a buffer, one or more chemical stabilizers; and one or more surfactants, wherein the reagent formulation is not in contact with the working electrode.

Still another aspect of the present invention is a biosensor having a pretreatment reagent upstream of the reagent along a microfluidic channel. In one embodiment, the biosensor comprises (a) a microfluidic channel defining a sample inlet; (b) a reagent disposed along the microfluidic channel, the reagent comprising at least an ester-containing substrate with an electrochemical mediator, a buffer, one or more chemical stabilizers; and one or more surfactants, the reagent being electrochemical reactive with LE; (c) at least one pre-treatment reagent along the microfluidic channel between the sample inlet and the reagent, and comprising a concentrated pH neutralization buffer, an ascorbate neutralizer, and optionally a surfactant either separate or in combination.

Another aspect of the present invention as a method of using the biosensor disclosed herein. In one embodiment, the method comprises (a) inserting the biosensor into a reader; (b) applying a sample of biological fluid to the biosensor sensor comprising a reagent; (c) measuring voltage and/or current output of an electrochemical reaction between the reagent and the sample, and (d) correlating the output to a level of LE.

Still other aspects and embodiments of the present invention will be obvious to those of skill in the art in light of this disclosure.

### BRIEF DESCRIPTION OF FIGURES

FIG. 1A is a perspective view of one embodiment of an assembled biosensor for the detection of leukocyte esterase in synovial fluid.

FIG. 1B is an exploded view of biosensor from FIG. 1A.

FIG. 2 shows a calibration curve of PLE spiked into artificial synovial fluid samples.

FIG. 3 is a graph showing clinical results of detection of leukocyte esterase in clinical synovial fluid samples.

FIG. 4 is a graph showing mid-term stability test sensors with α-cyclodextrin and 0.1% trehalose at 20° C.

FIG. 5 is a graph showing effect of CHAPS concentration and incubation time on extent of lysis.

FIG. 6 is a graph showing effect of non-ionic surfactants on WBC lysis in wet assay format.

FIG. 7 is a graph showing lysis efficiency of test sensors with and without non-ionic surfactant.

FIG. 8 is a top view of one embodiment of a screen-printed electrode sensor for the detection of leukocyte esterase in urine.

FIG. 9 is a calibration curve of WBCs spiked into artificial urine samples.

FIG. 10 is a graph showing a reduction peak height with and without pre-condition step urine samples.

FIG. 11 is a graph showing enhancement of leukocyte esterase detection with myoinositol.

### DETAILED DESCRIPTION

In the following paragraphs, the present invention will be described in detail by way of example with reference to the attached drawings. Throughout this description, the preferred embodiment and examples shown should be considered as exemplars, rather than as limitations on the present invention. As used herein, the “present invention” refers to any one of the embodiments of the invention described herein, and any equivalents. Furthermore, reference to various feature(s) of the “present invention” throughout this document does not mean that all claimed embodiments or methods must include the referenced feature(s).

### Low pH Buffer

Enzymes have high substrate selectivity, a characteristic which can be used to support the specificity of an in vitro diagnostic test. It is common practice for an in vitro diagnostic test to utilize an enzyme to measure or detect the presence of its substrate as an analyte of interest. But it is less common for an in vitro diagnostic test to measure or detect the presence of an enzyme, such as leukocyte esterase (LE), based on its hydrolysis of a specific substrate as an indicator for the assay. This poses the unique challenge of requiring a stabilized chemical substrate for long-term storage that is also capable of being rapidly hydrolyzed upon introduction of a test sample containing the target enzyme.

To prepare a dry reagent for an in vitro diagnostic test involving an enzyme-catalyzed chemical reaction, the traditional approach is to introduce a chemical reagent formulation in a suitable buffer that will ensure the enzymatic reaction of the assay occurs at the optimal pH for maximal enzyme activity. For LE, maximal enzyme activity is observed at neutral pH (pH 7-8). However, a chemical substrate with a simple ester can spontaneously hydrolyze, and the rate of hydrolysis is proportional to the amount of base present to be consumed in the reaction. As such, the rate of hydrolysis of the ester substrate is theoretically reduced 1,000- to 100,000-fold at pH between 2 and 4 compared to pH 7. Further reducing the pH below 2 can, conversely, lead to acid-catalyzed hydrolysis of the substrate. When compared experimentally to incubation at pH 8, the peak current in square wave voltammetry from incubation of pig liver esterase with an ester-containing substrate for 5 minutes was reduced by about 40% at pH 6 and 75% at pH 3. Although a low pH of 2 to 4 is optimal for stabilizing the chemical ester-containing substrate, LE enzyme cannot function effectively at a low pH.

Certain embodiments of the present invention are directed to screen-printed electrode sensors for the detection of LE, wherein an ester-containing substrate with an electrochemical mediator is stabilized in a chemical reagent formulation containing both a low pH buffer to reduce the rate of ester hydrolysis and one or more chemical stabilizers. Further, the reagent formulation is dried down as a stable reagent cake within a microfluidic flow cell to be resuspended by the test sample. The chemical reagent formulation is designed such that the pH of the assay solution resulting from resuspension of the dry reagent cake is rapidly restored to neutral pH by the natural buffering capacity of the biological fluid sample to allow immediate reversal of the conditions intended for stabilization of the ester-containing substrate during storage to conditions for optimal enzyme-catalyzed hydrolysis of the substrate. In some embodiments, an in vitro diagnostic test for LE containing a reagent formulation with an acetate buffer at a pH of about 3 significantly reduced background noise compared to an acetate buffer at a pH of about 5 or 6 by preventing non-specific hydrolysis, without reducing the overall signal of the assay thereby boosting the signal to noise of the assay. Compared to a reagent formulation with a glycine or citrate buffer, an acetate buffer also demonstrated rapid filling of the test sample within a passive microfluidic flow cell, likely due to the impact of the buffer reagent on the hydrophilic surface of the flow cell. In some embodiments, an in vitro diagnostic test for LE with a reagent formulation with a low pH glycine buffer demonstrated maximal signal to noise compared to a low pH acetate buffer or low pH citrate buffer.

In some embodiments, the test sample is periprosthetic synovial fluid which inherently is a neutral pH buffer and can directly buffer the reaction pH upon contact and resuspension of the chemical reagent formulation. In some embodiments, the test sample is urine which has a variable pH from 4 to 9. The urine test sample should contact and resuspend a dried pre-treatment reagent containing a concentrated buffer, such as phosphate buffer (250 mM to 1000 mM) to correct the pH of the test sample before it comes into contact with and resuspends the ester-containing reagent formulation to ensure restoration of a neutral pH for the enzyme-catalyzed chemical reaction. A phosphate buffer (250 mM) was more effective than a HEPES buffer (250 mM) at achieving target buffer pH.

In one embodiment, the present invention relates to a biosensor for the detection of LE with a reagent formulation comprising: (a) an ester-containing substrate with an electrochemical mediator; (b) a buffer having a pH sufficiently low to inhibit the hydrolysis of the substrate prior to introduction of a test sample having a higher pH; (c) one or more chemical stabilizers; and (d) one or more surfactants.

As mentioned above, in one embodiment, the ester-containing substrate is deposited in an acidic buffer with a pH between 2 and 4. In one embodiment, combining the test sample with the reagent formulation on the sensor increases the pH of the reagent formulation to that of the test sample, or near the test sample's pH. In one embodiment, the ester-containing substrate in acidic buffer is contacted with a test sample with a neutral ‘physiologic’ pH to restore normal enzyme function. In one embodiment, the ester-containing substrate and neutral pH test sample are contacted within a microfluidic channel.

In one embodiment, the buffer has a pH of less than 4. In one embodiment, the buffer is one of acetate, glycine, or citrate. In one embodiment, the buffer is acetate or glycine with a pH of between 2 and 4. In one embodiment, the final concentration of the ester-containing substrate is about 1 mM to about 50 mM, or about 10 mM to about 40 mM, or 20 mM to about 30 mM.

### WBC Lysis: Surfactant

LE is an enzyme contained within WBCs that is released upon cell lysis. Although WBCs will eventually lyse spontaneously or with freeze-thaw, testing fresh samples immediately after collection requires efficient and complete cell lysis. Inadequate lysis leads to under-recovery of LE from a test sample since esterase enzyme is not released from unlysed cells. A proper lysis reagent is required that can rapidly lyse cells, usually containing one or more surfactants. However, the introduction of surfactants to in vitro diagnostic tests can have a major impact on assay performance, and sometimes unexpected or unintended consequences due to changes in surface energy of a microfluidic flow cell, undesirable effects on screen-printed electrodes, or denaturation or inactivation of enzymes that take part in the enzyme-catalyzed chemical reaction. The amount and type of surfactant needed to effectively lyse WBCs can depend on the incubation time and the reagent buffer conditions.

The zwitterionic surfactant CHAPS (3-[(3-Cholamidopropyl)dimethylammonio]-1-propanesulfonate) is commonly used in diagnostic assays because it is generally non-denaturing and maintains enzyme function, it is chemically stable, and it also helps solubilize and resuspend reagent formulation. A concentration of 0.25% to 2% CHAPS is typically sufficient to completely lyse WBCs in a diagnostic assay. For an in vitro diagnostic for LE, the addition of about 0.25% to about 5% CHAPS directly into a test sample demonstrated rapid lysis of WBCs at all concentrations tested in 2 minutes or less with no significant change in background signal (assay with no WBCs present). However, upon depositing and drying CHAPS at concentrations from 0.25% to 1% w/v in a reagent formulation onto the surface of a microfluidic flow cell to be resuspended by the test sample, insufficient lysis was observed with CHAPS concentrations from 0.2% to 1% with incubations of less than 10 minutes. This is likely due to slow resuspension and resolubilization of the CHAPS reagent from the dry reagent cake into the test sample, which required at least 10 minutes. A 1% concentration of CHAPS also non-specifically increased the background signal of the assay (tested with no WBCs present). Unexpectedly, CHAPS alone was not sufficient as a lysis agent. Similar to CHAPS, non-ionic surfactants are mild and non-denaturing, and can be suitable as lysis agents in diagnostic applications. Two non-ionic polyethylene oxide surfactants, Triton X-100 and Igepal CA-630, spiked directly into a test sample demonstrated rapid lysis of WBCs within 2 minutes at concentrations of 0.5% w/v or less, albeit with a concentration-dependent increase in background noise, while a third non-ionic polyethylene oxide surfactant Brij-35 did not demonstrate any lysis after 2 minutes. The maximum WBC lysis efficiency relative to the increase in background noise was achieved with both Triton X-100 and Igepal CA-630 at a concentration of about 0.1% w/v. Unexpectedly, signal to noise was further improved by combining both Triton X-100 and Igepal CA-630 at their maximal effective concentrations, which indicates surprisingly that their contributions to background noise may not be additive despite their similarity, and optimal WBC lysis is achieved with a combination of these non-ionic surfactants. Unlike CHAPS for which effective lysis was lost upon drying of the surfactant into the chemical reagent formulation, the chemical reagent formulation containing the non-ionic surfactants Triton X-100 and Igepal CA-630 remained effective within 2 minutes or less upon resuspending from a dried reagent cake. This may reflect superior resuspension and resolubilization of non-ionic surfactants. Anionic surfactants, such as DOSS and SDS, were also tested and found to be not suitable due to large increases in background noise from electrochemical interference.

(Triton X-100, Triton X-114, and Igepal CA-630 are well known surfactants. Each has the same chemical name octylphenoxypolyethoxyethanol, which is a repeating unit:

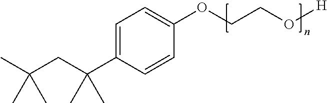

The difference among them is the number of repeating units, which affects hydrophilicity. Specifically, Triton X-100 and Igepal CA-630 have the formula C14H22O(C2H4O)n, where n is around 9 to 10. Both of these have the same number of repeats. The average n is slightly lower for Triton X-100 which is very slightly more hydrophilic than Igepal CA-630. Triton X-114 has the formula C14H22O(C2H4O)n, where n is around 7 to 8. Triton X-114 has smaller chains and is less hydrophilic.)

In one embodiment, the present invention relates to a biosensor for the detection of LE with a reagent formulation comprising: (a) an ester-containing substrate with an electrochemical mediator; (b) a buffer; (c) one or more chemical stabilizers; and (d) one or more surfactants, at least one being non-ionic, wherein the one or more surfactants is at least 0.1% w/v or 0.05% and 0.2% of the reagent formulation.

In one embodiment, the non-ionic surfactant is a polyethylene oxide surfactant. In one embodiment, the one or more surfactants comprises at least two polyethylene oxide surfactants. In one embodiment, the non-ionic surfactant is one or more of Triton X-100, Triton X-114, IgepalCA-630, or Digitonin. In one embodiment, the two non-ionic polyethylene oxide surfactants are Triton X-100 and Igepal CA-630. In one embodiment, the non-ionic surfactant is a steroidal saponin surfactant. In one embodiment, the non-ionic steroidal saponin surfactant is digitonin.

In one embodiment, the surfactant is at least one of Triton X-100 or Igepal CA-630, at a concentration of between 0.01% and 1% w/v, or 0.05% and 0.2% weight to volume. In one embodiment, the non-ionic surfactant is a combination of Triton X-100 and Igepal CA-630, each between 0.05% and 0.2% weight to volume.

In one embodiment, the surfactant is a zwitterionic surfactant. In one embodiment, a zwitterionic surfactant is combined with one or more non-ionic surfactants. In one embodiment, the zwitterionic surfactant is 3-[(3-cholamidopropyl)dimethylammonio]-1-propanesulfonate (“CHAPS”). In one embodiment, CHAPS is between about 0.01% and 10%, or about 0.1-5%, or about 1-2% weight to volume.

Reagent Formulation does not Contact Electrodes

For in vitro diagnostic tests using screen-printed electrodes, the reagent formulation is often deposited directly onto the working electrode to bring the enzyme-catalyzed reaction as close as possible to the detection element. For an in vitro diagnostic for LE, test sensors wherein the ester-containing reagent formulation was deposited and dried directly on the working electrode demonstrated unexpectedly poor performance with significant distortion of observed cyclic voltammograms. This distortion, which likely reflects electrode fouling, significant impaired the performance of the test, resulting in poor sensitivity and signal to noise. Test performance was restored by depositing and drying the reagent away from the working electrode. In some embodiments, the reagent is deposited co-facially directly across from the working electrode within a microfluidic flow cell. In other embodiments, the reagent formulation is deposited upstream from the working electrode within a microfluidic flow cell but on the same surface (co-planar) as the working electrode. The advantage of co-facial deposition of the dried reagent formulation is that the test sample can contact both the reagent formulation and working electrode at the same time since they are physically adjacent from one another. This maximizes the time for resuspension of the reagent formulation. With coplanar deposition, the reagent formulation and working electrode are physically separated on the same surface of the microfluidic flow cell. Reagent resuspension must occur more quickly as the test sample passes over the dried reagent formulation upstream in the microfluidic channel before advancing within the microfluidic flow cell to the working electrode for analysis.

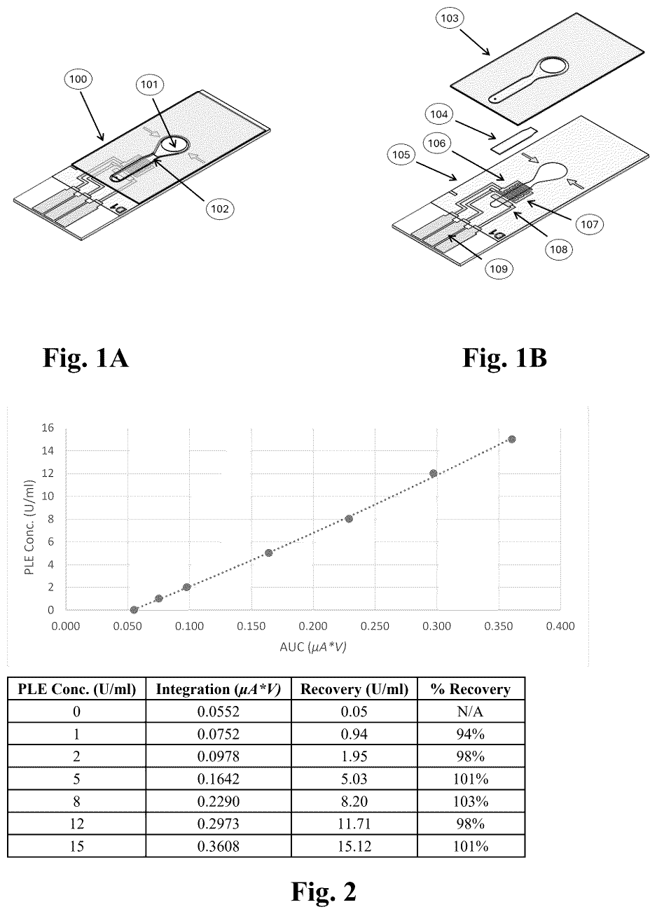

Referring to FIGS. 1A and 1B, one embodiment of the biosensor 100 is shown in perspective view and exploded view, respectively. In this embodiment, the biosensor 100 is configured to receive a sample at a sample inlet 101. The sample then is communicated along microfluidic flow cell 102 such that it contacts the reagent formulation 104. Depending upon the level of LE in the sample, there will be an electrochemical reaction between the sample and the reagent formulation producing an electrical voltage/current at the working electrode 107, which is then conducted to instrument contact pads 109, along with signals from a counter electrode 106 and reference electrode 108. A reader (not shown) is then used to interpret electrical signals at the instrument contact pads.

In one embodiment, the biosensor 100 comprises (a) at least one working electrode 107 on a surface 105; (b) a reagent formulation 104 comprising at least an ester-containing substrate with an electrochemical mediator, a buffer, one or more chemical stabilizers; and one or more surfactants, (c) wherein the reagent formulation 104 is not in contact with the working electrode 107.

In one embodiment, the surface 105 with the working electrode forms the base of a microfluidic flow cell 102 and is adjacent to a second surface 103 that forms the top of the microfluidic flow cell 102. In one embodiment, the reagent formulation is deposited and dried co-facially on the second surface adjacent to the surface containing working electrode so as to avoid contact between the reagent formulation and the working electrode. When the fluid fills the channel, it resuspends the dried reagent into the fluid sample.

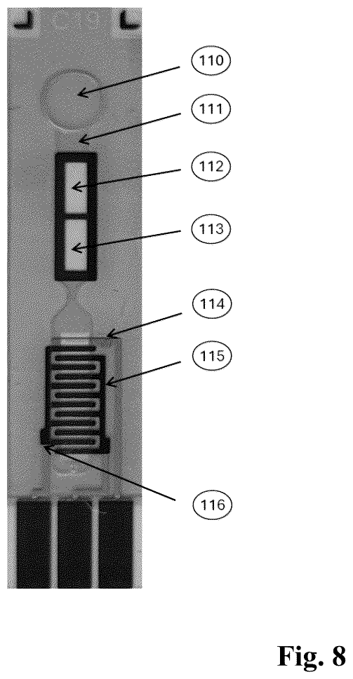

Referring to FIG. 8, in one embodiment, the reagent formulation 113 is dried within the microfluidic channel 111 upstream of the working electrode 115 so as not to be in contact with the working electrode.

In one embodiment, the working electrode is an interdigitated electrode. In one embodiment, the working electrode is a carbon ink interdigitated electrode.

In one embodiment, the biosensor is screen printed, although other manufacturing techniques will be obvious to those of ordinary skill in the art in light of this disclosure.

### Ascorbate Neutralization—Pretreatment

In addition to the variable pH of urine, urine specimens can sometimes contain large amounts of the chemical reducing agent ascorbate which can be a significant interferent in an in vitro diagnostic test. Ascorbate levels of 10 to 50 mg/dL are typical of individuals taking ascorbate supplements. For an electrochemical in vitro diagnostic test for LE, the percent recovery of LE was significantly reduced in the presence of ascorbate, with a bias of 23% at 25 mg/dL and 42% at 50 mg/dL. Ascorbate must be neutralized by a chemical neutralizer to eliminate bias without itself causing assay bias in an electrochemical assay. In some embodiments, a large excess of sodium iodate (80 mM) is incorporated into a pre-treatment reagent to neutralize ascorbate in a urine sample before it comes into contact and resuspends a reagent formulation. Neutralization with iodate improved percent recovery, with a bias of only 3% at a 25 mg/dL ascorbate and 10% at 50 mg/dL ascorbate level. In other embodiments, a small excess of chloramine can replace iodate as a neutralizing agent. Ascorbate tablets are known to neutralized chloramine, a water disinfectant similar to chlorine, in pools, and likewise, chloramine can also neutralized ascorbate. Hydrogen peroxide was not an effective ascorbate neutralizer.

In one embodiment, the biosensor comprises one or more pre-treatment reagent(s) containing a concentrated pH neutralization buffer, an ascorbate neutralizer, and optionally a surfactant either separate or in combination. Referring to FIG. 8, another embodiment of a biosensor of the present invention is shown. In this embodiment, the biosensor 100 is configured to receive a sample at the sample inlet 110. Like the embodiment of FIG. 1, the sample then is communicated along microfluidic flow cell 111 such that it contacts the reagent formulation 113. However, in this embodiment, the sample first comes into contact with a pretreatment reagent formulation 112 upstream of the reagent formulation. Specifically, in this embodiment, the pre-treatment reagent formulation 112 is dry deposited within the microfluidic channel 111 upstream from reagent formulation so that the pre-treatment reagent formulation is resuspended into a biological sample before it contacts the reagent formulation. (In this particular embodiment, unlike the embodiment of FIG. 1, the reagent formulation is deposited co-planar onto bottom surface of microfluidic flow cell.) As with the embodiment of FIG. 1, any electrochemical reaction between the sample and the reagent formulation produces an electrical voltage/current at the working electrode 116, which is then conducted to instrument contact pads, along with signals from a counter electrode 115 and reference electrode 114. A reader is then used to interpret electrical signals at the instrument contact pads.

In one embodiment, the pre-treatment reagent contains a concentrated pH neutralization buffer. In one embodiment, the pH neutralization buffer of the pre-treatment reagent contains concentrated phosphate buffer solution. In one embodiment, the pH neutralization buffer of the pre-treatment reagent is about 250 mM to 1000 mM phosphate buffer solution.

In one embodiment, the pre-treatment reagent contains an ascorbate neutralizer. In one embodiment, the ascorbate neutralizer of the pre-treatment reagent is chloramine. In one embodiment, the chloramine is present in an amount of 1 to 10 mM, or 2 to 5 mM.

In one embodiment, the pre-treatment reagent contains a surfactant. In one the surfactant of the pre-treatment reagent is approximately 1% CHAPS.

### Stabilizer

In one embodiment, the reagent also includes a stabilizer comprising at least one of a carbohydrate or saccharide. In one embodiment, the saccharide is a sugar trehalose is about 0.1% weight to volume. In one embodiment, the trehalose is about 200 mol % to 400 mol % of the buffer. In one embodiment, the stabilizer is a cyclic oligosaccharide. In one embodiment, the cyclic oligosaccharide is a cyclodextrin. In one embodiment, the cyclodextrin is about 100 mol % to 200 mol % of the ester-containing substrate. In one embodiment, the cyclodextrin is alpha-cyclodextrin. In one embodiment, the alpha-cyclodextrin is about 100 mol % to 200 mol % of the ester-containing substrate present. In one embodiment, the alpha-cyclodextrin is 110 mol % of the ester-containing substrate.

### Ester-Containing Substrate

The substrate may be any substrate disclosed in U.S. application Ser. Nos. 16/087,411 and 16/145,014. In one embodiment, the ester-containing substrate comprises but is not limited to phenol, hydroquinone, or a derivative thereof. In one embodiment, the ester-containing substrate can be hydrolyzed by the enzyme LE. In one embodiment, the ester-containing substrate is:

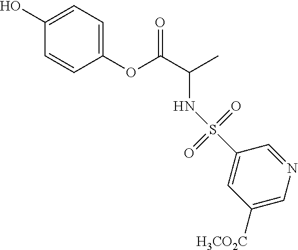 [SMILES: COC(=O)c1cncc(S(=O)(=O)NC(C)C(=O)Oc2ccc(O)cc2)c1]

### Channel Configuration

The microfluidic channel configuration is important to ensure proper flow of the sample into the regent and those of ordinary skill in the art in light of this disclosure will be able to optimize the channel for a given application. In one embodiment, the inlet channel is fluted. In one embodiment, the microfluidic channel contains a pin-sized hole at its base that forms a vent stop. In one embodiment, the microfluidic channel is formed with die cut pressure sensitive adhesive.

### Method of Diagnosing Infection

Another aspect of the invention is a method of using the biosensor disclosed herein for diagnosing infection. In one embodiment, the method comprises (a) inserting the biosensor into a potentiostat; (b) applying a sample of biological fluid to the sensor comprising a reagent; (c) measuring the voltage and/or current output of an electrochemical reaction between the reagent and the sample, and (d) correlating the output to the level of LE. In one embodiment, the biological fluid sample is one of synovial fluid, cerebrospinal fluid, or urine.

In one embodiment, the voltage and/or current output of an electrochemical reaction is measured using an electrochemical technique. In one embodiment, the electrochemical technique is square wave voltammetry or chronoamperometry. In one embodiment, the electrochemical technique is square wave voltammetry. In one embodiment, the electrochemical technique is performed about 1 to 5 minutes after the biological fluid is applied to the screen-printed electrode.

In one embodiment, detecting LE in joint fluid is determined based on the oxidation current peak. In one embodiment, the electrochemical detection of LE in joint fluid is determined based on the integration of area under the oxidation current peak. In one embodiment, the electrochemical detection of LE in joint fluid is determined based on the current under the oxidation peak between 0 and 1 Volts. In one embodiment, the electrochemical detection of LE in joint fluid is determined based on the current under the oxidation peak between 0.1 and 0.7 Volts to avoid interference from silver contamination.

In one embodiment, the electrochemical detection of LE in joint fluid based on the oxidation current includes an overpotential with a scan starting from a negative potential to a positive potential. In one embodiment, the electrochemical detection of LE in joint fluid based on the oxidation current includes a scan from −0.1 to +1 Volts.

In one embodiment, the electrochemical detection of LE in urine is determined based on the reduction current peak to avoid interference. In one embodiment, the electrochemical detection of LE in urine is determined based on the integration of area under the reduction current peak.

In one embodiment, the electrochemical detection of LE in urine based on the reduction current peak includes an overpotential with a scan starting from a positive potential to a negative potential. In one embodiment, the electrochemical detection LE in urine based on the reduction current peak includes a scan from +0.8 to −0.6 Volts. In one embodiment, the electrochemical detection LE in urine based on the reduction current peak includes a scan from +0.2 to −0.6 Volts. In one embodiment, the electrochemical detection of LE in urine based on the reduction current peak requires a pre-condition step to fully oxidize hydroquinone immediately before starting an electrochemical measurement. In one embodiment, the electrochemical detection of LE in urine based on the reduction current peak requires a pre-condition step that comprises applying a positive potential of approximately 0.5 to 1 volt to fully oxidize hydroquinone immediately before starting an electrochemical measurement. In one embodiment, the electrochemical measurement for the detection of LE in urine based on the reduction peak is run with a scan rate of greater than or equal to 20 Hz to limit the spontaneous reduction of hydroquinone after the pre-condition step.

In one embodiment, the level of LE in a synovial fluid sample is calibrated based on a calibrator matrix spiked with porcine liver esterase. In one embodiment, the calibration matrix is comprised of pooled negative clinical periprosthetic synovial fluid samples. In one embodiment, the calibrator matrix is spiked with porcine liver esterase between 0 and 30, or between 5 and 20 U/ml for calibration.

In one embodiment, a test is reported as negative when the calibrated level of LE is approximately less than or equal to 6 or 4 or 2 U/ml. In one embodiment, a test is reported as positive when the calibrated level of LE is approximately greater than or equal to 6, or 8, or 10 U/ml. In one embodiment, a test is reported as intermediate when the calibrated level of LE is greater than the negative threshold but below the positive threshold.

### EXAMPLES

Test sensors were prepared for detection of LE in synovial fluid from a periprosthetic hip or knee joint. In brief, screen-printed electrochemical sensors for the detection of LE were prepared as follows: (1) sheets of polyester base film were screen-printed with carbon, silver, silver chloride, and dielectric inks to form electrodes and cut into bands, (2) hydrophilic coated polyester film and pressure-sensitive adhesive film were also cut into bands and laminated together to form the top of a flow cell, (3) a reagent formulation was prepared containing an ester-containing substrate, acetate buffer pH 3.5, chemical stabilizers α-cyclodextrin and trehalose, non-ionic surfactants Triton X-100 and Igepal CA-630, and zwitterionic surfactant CHAPS hydrate (Table 2), and, subsequently, the reagent formulation was deposited and dried onto the hydrophilic coated surface of the flow cell, and finally (4) the bands of film containing screen-printed electrodes and hydrophilic flow cells containing dried reagent formulation were laminated together and singulated into individual test sensors that were sealed in desiccated pouches (FIG. 12). In the primary embodiment, the ester-containing substrate is 4-Hydroxyphenyl (N-(3-(methoxycarbonyl)pyridine-5-sulfonyl)-L-alaninate. Each molecule of ester-containing substrate releases one molecule of hydroquinone upon ester hydrolysis, which is detected by the screen-printed electrodes. A synovial fluid sample is applied as a droplet and enters the flow cell by passive capillary action to resuspend the dried reagent formulation on the surface of the flow cell, advancing passively until it reaches a vent stop at the end of the flow cell. After a brief incubation to allow for enzyme-specific hydrolysis of the chemical substrate by LE, the activity of the enzyme is detected.

In one example, artificial synovial fluid samples with 3% bovine serum albumin in Phosphate Buffered Saline (PBS) solution were spiked with porcine liver esterase (PLE) at 7 different concentration levels (0, 1, 2, 5, 8, 12, and 15 Units/ml). Each sample was tested in replicates of 5 after incubation for 2 minutes, and then an electrochemical measurement was conducted with square wave voltammetry. Analysis was performed with integration of the square wave voltammetry measurements from 0.1 to 1.0 Volts (V). The average integration of current in the potential range from 0.1 to 1 V (AUC) was plotted for each sample against its corresponding PLE concentration generating a polynomial line with an R² of 0.999. The PLE concentration of the 7 samples was extrapolated to determine spike-recovery. All spike-recovery levels were within +6% of the expected concentration. The test sensors demonstrated good linearity and recovery of artificial synovial fluid spiked samples from 0 to 15 U/ml activity level of PLE (FIG. 2), with recovery within 10% of the expected value for all samples tested and a R² of greater than 0.95. The limit of blank and limit of detection were both less than 0.5 U/ml.

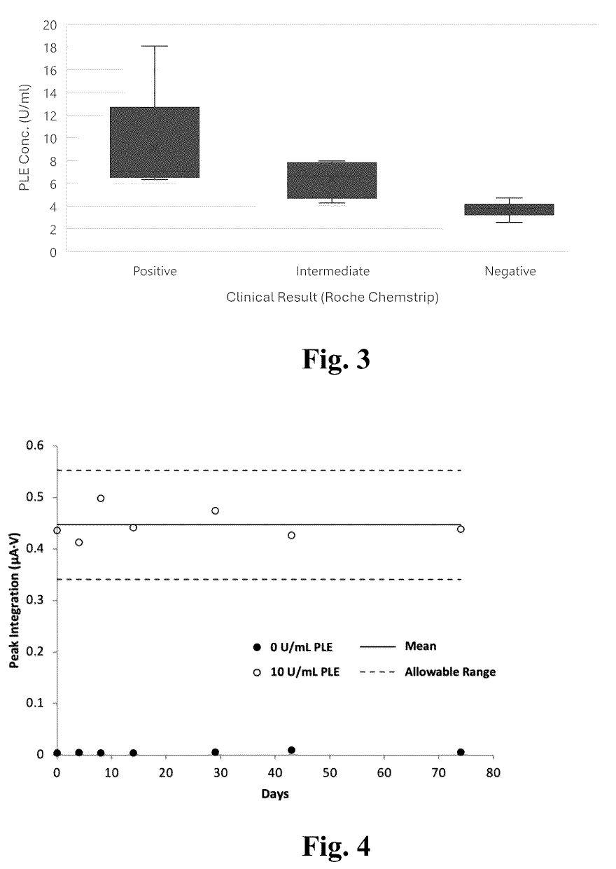

Additionally, synovial fluid samples collected from periprosthetic hip or knee joints were randomly selected with positive (2+), intermediate (1+), and negative (neg/trace) test results as determined by testing with the commercially available Roche Chemstrip® dipstick test. Frozen samples were allowed to defrost at room temp for at least 30 minutes prior to application to the test sensors. Each clinical sample was tested after incubation for 2 minutes, and then taking measurement with square wave voltammetry. Data was analyzed based on an integration from 0.1 to 1 V, and activity levels were calculated from a calibration curve relative to artificial synovial fluid samples. Calibrated results showed good differentiation of Positive, Intermediate, and Negative clinical samples based on the Roche Chemstrip designations (FIG. 3).

TABLE 1
Theoretical reagent concentrations after
complete resuspension in synovial sample

| Component | Amount | Units |
|---|---|---|
| CHAPS Hydrate | 0.03 | w/v % |
| Sodium Acetate | 3.18 | mM |
| Trehalose | 0.29 | w/v % |
| Triton X-100 | 0.05 | w/v % |
| Igepal CA-630 | 0.06 | w/v % |
| α-Cyclodextrin | 0.62 | w/v % |
| Ester Substrate | 6.00 | mM |

The optimal pH for detection of LE was assessed in an experiment with a wet assay. Research-grade screen-printed electrode sensors (DropSens, Metrohm, Switzerland) were spiked with citric/phosphate buffer solutions of various pH levels (pH 3 to 8) containing the ester-containing substrate and 10 U/ml PLE. Test samples were incubated for 5 minutes before performing square wave voltammetry (−0.5 to +1 V, frequency 1 Hz). The hydrolysis of the ester-containing substrate was inhibited at acidic pH, thereby reducing detection of LE (Table 2). Thus, a neutral physiologic pH is the optimal reaction condition within the flow cell for hydrolysis of the ester-containing substrate and detection of LE.

TABLE 2
Peak current measurement with square wave voltammetry by assay

| pH | 3 | 4 | 5 | 6 | 7 | 8 |
|---|---|---|---|---|---|---|
| μA | 1.65 | 1.77 | 2.76 | 4.12 | 6.04 | 6.73 |

Despite a neutral pH being the optimal reaction condition for substrate hydrolysis in the presence of esterase enzyme, the reagent formulation must also balance the need to stabilize the chemical substrate and prevent non-specific hydrolysis either during storage or with wetting by a test sample. Test sensors were prepared with reagent formulations using acetate buffer at pH 3.3, pH 5.0, and pH 6.0. Test sensors were evaluated with negative (0 U/ml PLE) and positive (10 U/ml PLE) artificial synovial fluid samples to assess background noise (esterase activity with negative test sample from non-specific hydrolysis) and signal to noise (ratio of esterase activity with positive test sample versus negative test sample), respectively, with each reagent pH condition. Test sensors prepared with acetate buffer pH 3.3 had significantly lower background noise, as determined based on esterase activity with a negative test sample of 0.047±0.008 μA*V compared to esterase activity of 0.091±0.014 μA*V with acetate buffer pH 5.0 and esterase activity of 0.094±0.005 μA*V with acetate buffer pH 6.0. Test sensors with acetate buffer pH 3.3 also had a significantly greater signal to noise, as determined based on 9.4-fold greater activity with a positive test sample versus a negative test sample compared to 4.7-fold with acetate buffer pH 5.0 and 5.6-fold with acetate buffer pH 6.0. Although the acidic pH of the buffer stabilized the reagent and reduced background noise by slowing the rate of non-specific hydrolysis, it, paradoxically, had no significant impact on the esterase activity of a positive test sample, thereby improving signal to noise. With the relatively large volume and buffering capacity of the synovial fluid test sample relative to the reagent formulation, the test sample rapidly restores the neutral pH of the reagent upon resuspension, thereby restoring optimal esterase enzyme activity. Thus, the result is improved stability of the ester-containing substrate with a concomitant increase in assay signal to noise of the assay using a buffer with pH 2-4.

In another example, test sensors were prepared using glycine buffer, pH 3.4 or citrate buffer, pH 3.5 compared to acetate buffer, pH 3.5 for the reagent formulation dried onto the hydrophilic surface of the flow cell. With application of a 5 μl droplet of artificial synovial fluid, test sensors prepared with acetate buffer rapidly filled the hydrophilic flow cell in less than 10 seconds with good reproducibility (CV %<10%). Despite demonstrating good signal to noise, test sensors prepared with glycine or citrate buffer had extremely poor passive fill characteristics, presumably due to the impact of the reagent on the surface energy of the hydrophilic surface of the flow cell. Test sensors prepared with glycine buffer slowly filled the flow cell, requiring up to 1 minute or more for complete fill, and demonstrated poor reproducibility (CV % 22%). Test sensors prepared with citrate filled extremely slowly and did not completely fill the flow cells with a 5 μl droplet.

The ester-containing substrate in the reagent can hydrolyze spontaneous in the presence of water or moisture. Chemical stabilizers, such as cyclodextrins and trehalose, can be used to form a stable dry reagent cake to protect the ester-containing substrate. Test sensors were prepared with a reagent formulation containing a molar excess of α-cyclodextrin and varying concentrations of trehalose (0.0%, 0.01%, 0.05%, or 0.1%). During a short cure phase at 40° C., the signal to noise of test sensors prepared without trehalose or with 0.01% trehalose was reduced by 32% and 47%, respectively, whereas the decrease in signal to noise was only 13% and 20%, respectively, for test sensos prepared with 0.05% or 0.1% trehalose. Test sensors prepared with a combination of α-cyclodextrin and trehalose as chemical stabilizers demonstrated long-term stability for at least 6 months in real time (20° C.) and accelerated (40° C.) conditions (mid-term stability shown in FIG. 4).

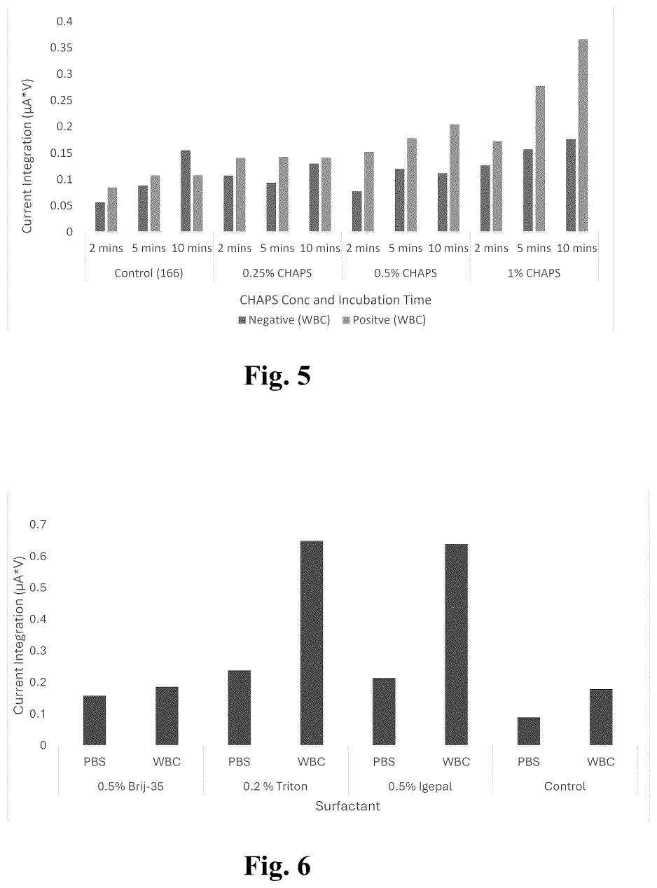

LE is an enzyme released by white blood cells. Therefore, white blood cells must be completely lysed to release the enzyme contents to be detected in an assay. Surfactants are typically used as chemical lysis agents, but the use of surfactants in in vitro diagnostics can be complicated by the effects of surfactants on enzymes or proteins involved in the assay chemistry and/or the impact of surfactants on screen-printed electrodes. CHAPS (3-[(3-Cholamidopropyl)dimethylammonio]-1-propanesulfonate) is a zwitterionic non-denaturing surfactant that is commonly used as a wetting agent or lysis agent in diagnostic assays. Low concentration CHAPS (0.01%) was used as a wetting agent but, unexpectedly, was completely ineffective as a lysis agent. However, higher concentrations of CHAPS were evaluated for suitability as a lysis agent. Human white blood cells isolated from fresh whole blood obtained by phlebotomy were spiked into an artificial synovial fluid matrix to test for the extent of WBC lysis. Test sensors prepared for the detection of LE (ester-containing substrate, sodium acetate, α-cyclodextrin, trehalose, and 0.01% CHAPS) were evaluated in a wet assay experiment using human WBCs spiked into PBS compared to human WBCs spiked into PBS containing 5% CHAPS. The esterase activity of spiked WBCs in a PBS matrix containing 5% CHAPS was 4.3-fold greater than the activity of spiked WBC in PBS with no CHAPS. In a second experiment, test sensors were spiked with neat plasma or plasma containing 5% CHAPS with no WBCs present. The esterase activity of plasma with 5% CHAPS was 1.6-fold greater than the activity of neat plasma. Thus, although 5% CHAPS could efficiently induce WBC lysis in a wet assay, it also significantly increased background noise in an artificial synovial fluid matrix. In a second wet assay experiment, CHAPS concentrations from 0% to 5% spiked directly into PBS matrix were tested with and without human WBCs after an incubation time of 2 minutes. There was no increase in esterase activity when human WBCs were spiked into a neat PBS matrix or PBS matrix with 0.05% CHAPS. However, the background noise increased almost 2-fold with the addition of 0.05% CHAPS to PBS. An increase in esterase activity of 1.8 to 3.3-fold was observed when human WBCs were spiked into a PBS matrix with a CHAPS concentration of 0.25% or greater, but there was no clear concentration-dependent increase in signal to noise with a concentration of CHAPS above 0.25%. Although signal to noise was increased with the addition of CHAPS, this was limited by the increased background noise with CHAPS. Finally, test sensors were prepared with increasing concentrations of CHAPS (0.25%, 0.5%, and 1%) added to the dry reagent formulation. Artificial synovial fluid samples were applied with and without spiked human WBCs, and measurements were conducted after incubation for 2, 5, and 10 minutes (FIG. 5). In a dry format, there was insufficient lysis at all CHAPS concentrations with incubation for 2 minutes and CHAPS concentrations of 0.25% or 0.5%. The extent of lysis improved with 1% CHAPS after a 10-minute incubation, likely due to slow re-solubilization of the CHAPS surfactant, but this also corresponded to an increase in background signal. Therefore, CHAPS was deemed not to be a suitable lysing agent. CHAPS concentrations of 0.01% to 0.1% in a dry reagent formulation can improve wetting while having no impact on lysis or background noise.

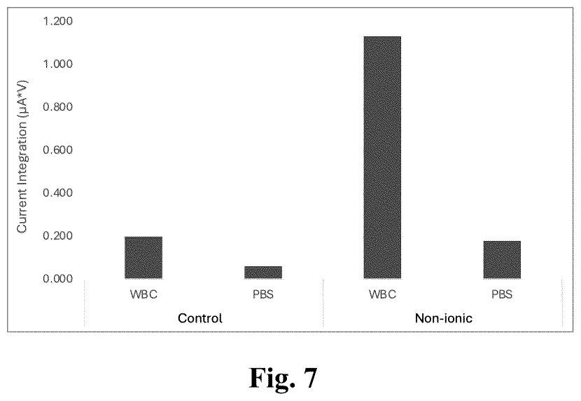

A series of surfactants were evaluated for suitability as a lysis agent. Anionic surfactants, such as dioctyl sulfosuccinate sodium salt (DOSS) and sodium dodecyl sulfate (SDS), significantly increased background noise and could not be considered. On the contrary, non-ionic polyethylene oxide surfactants (e.g., Triton X-100, Triton X-114, Igepal CA-630, Brij-35) and steroidal saponin surfactants (Digitonin) generally had less impact on background noise. In wet assay experiments, Triton X-100 and Igepal CA-640, but not Brij-35, demonstrated good WBC lysis after a 2-minute incubation, albeit at significantly higher concentrations than anticipated and with an increase in background noise, as determined by comparing esterase activity following spiking into artificial synovial fluid samples with and without WBCs (FIG. 6). Trials were conducted evaluating Triton X-100 and Igepal CA-640 at various concentrations ranging from 0.03125%-0.5% for Igepal CA-540 and from 0.0125%-0.2% for Triton X-100. The lysis efficiency was evaluated based on effectiveness at increasing esterase signal (Lysis Ratio) less the increase in background noise (Noise Ratio) based on a comparison to artificial synovial fluid with no surfactant (Table 3). The optimal lysis efficiency was achieved with 0.05-0.1% Triton X-100 and 0.0625-0.125% Igepal CA-620. Despite their similarities as non-ionic surfactants, a combination of Triton X-100 (0.05% w/v) and Igepal CA-640 (0.0626% w/v %) unexpectedly provided a further increase in lysis efficiency, with an almost 3-fold increase in signal to noise compared to test sensors prepared without non-ionic surfactant (FIG. 7).

TABLE 3
Effective lysis ratio, background ratio, and critical lysis ratio

| | Conc. | Spiked WBCs | NO WBCS | Lysis | | Lysis |
|---|---|---|---|---|---|---|
| Surfactant | (w/v %) | (μA*V) | (μA*V) | Ratio | Noise Ratio | Efficiency |
| None | N/A | 0.23 | 0.04 | N/A | N/A | N/A |
| Igepal | 0.03125% | 0.73 | 0.09 | 3.21 | 1.96 | 1.25 |
| Igepal | 0.0625% | 1.08 | 0.15 | 4.76 | 3.44 | 1.32 |
| Igepal | 0.125% | 1.35 | 0.19 | 5.97 | 4.17 | 1.80 |
| Igepal | 0.25% | 1.08 | 0.26 | 4.79 | 5.88 | −1.09 |
| Igepal | 0.5% | 1.21 | 0.22 | 5.36 | 4.96 | 0.40 |
| Triton X-100 | 0.0125% | 0.66 | 0.05 | 2.91 | 1.02 | 1.90 |
| Triton X-100 | 0.025% | 0.59 | 0.07 | 2.60 | 1.65 | 0.94 |
| Triton X-100 | 0.05% | 1.33 | 0.15 | 5.89 | 3.45 | 2.44 |
| Triton X-100 | 0.1% | 1.24 | 0.13 | 5.50 | 2.95 | 2.55 |
| Triton X-100 | 0.2% | 1.20 | 0.17 | 5.33 | 3.93 | 1.40 |

With common diagnostic tests utilizing screen-printed electrode test sensors, such as glucose test strips for use with a glucometer, the traditional approach is to deposit and dry reagent formulation directly onto the working electrode that performs an electrochemical measurement. This has certain advantages such as the potential for enhanced sensitivity and reaction time since the reagent formulation is in direct contact with the measurement surface. In one example, test sensors were prepared for detection of LE in synovial fluid by depositing the reagent formulation directly onto the carbon working electrode. Tests were conducted with artificial synovial fluid samples with 0, 1, 5, and 10 U/ml PLE with incubation for 5 minutes. Paradoxically and despite the close proximity of reagent formulation, test sensors with reagent formulation deposited directly onto the working electrode demonstrated poor sensitivity, with a limit of blank of 0.5 U/ml compared to a limit of blank of less than 0.1 U/ml for test sensors with reagent formulation deposited co-facially directly adjacent to the working electrode on the hydrophilic surface of the top film. Furthermore, depositing reagent formulation directly on the working electrode also dramatically degraded the signal to noise of the assay, with an almost 3-fold reduction in signal to noise with a 10 U/ml artificial synovial fluid sample (Table 4). Assay precision was similar or even improved with co-facial deposition (coefficient of variation [CV %] of 5.6%) versus direct working electrode deposition (CV % of 7.9%). This is surprising given the need for improved reagent resuspension with co-facial deposition, and the potential impact of the dried reagent formulation on the surface energy of the hydrophilic surface. Upon evaluating cyclic voltammograms, it was determined that reagent deposition directly onto the working electrode caused distortion of electrochemical transients which introduced significant background noise. It is an object of this invention to deposit reagent formulation co-facially to overcome this limitation.

TABLE 4
Performance of test sensors with deposition
on working electrode versus top film

| | Working | |
|---|---|---|
| Characteristics | Electrode | Top Film |
| LOB (U/ml) | 0.09 | 0.49 |
| S:N 1 U/ml | 2.5 | 1.3 |
| S:N 5 U/ml | 6.9 | 2.4 |
| S:N 10 U/ml | 9.7 | 3.3 |

Limit of Blank (LOB),
Signal to noise (S:N)

Test sensors were prepared for detection of LE in urine. In brief, screen-printed electrochemical sensors for the detection of LE were prepared as follows: (1) sheets of polyester base film were screen-printed with carbon, silver, silver chloride, and dielectric inks to form electrodes and reagent deposition zones, and then cut into bands, (2) polyester film (uncoated) and pressure-sensitive adhesive film were also cut into bands and laminated together to form the top of a flow cell, (3) a main reagent formulation was prepared containing an ester-containing substrate, glycine buffer pH 3.3, chemical stabilizers α-cyclodextrin and trehalose, non-ionic surfactants Triton X-100 and Igepal CA-630, myoinositol, and zwitterionic surfactant CHAPS hydrate and a pre-treatment reagent formulation was prepared containing sodium iodate, CHAPS hydrate, and myoinositol (Table 5), and, subsequently, the pre-treatment reagent formulation and main reagent formulation were each deposited and dried in reagent deposition zones formed by hydrophobic ink (with the pre-treatment reagent formulation positioned upstream from the main reagent formulation), and finally (4) the bands of film containing screen-printed electrodes and top film of the flow cell were laminated together and singulated into individual test sensors that were sealed in desiccated pouches (FIG. 8). The position of the pre-treatment reagent upstream from the main reagent allowed neutralization of pH and ascorbate in the urine sample before it contacts and resuspends the main reagent with the ester-containing substrate. The position of the reagent deposition zones upstream from the working electrode also prevented electrode fouling from deposition directly onto the working electrode. For test sensors designed to detect LE in synovial fluid, co-facial deposition of the main reagent on the hydrophilic surface of the flow cell localized the reagent formulation directly adjacent the working electrode, which allowed the test sample to continuously resuspend the reagent formulation throughout the entire incubation period to maximize resuspension. On the contrary, co-planar deposition of reagent formulation upstream from the working electrode limited the resuspension time and localized the reagent formulation further from the working electrode. Despite the advantages of co-facial deposition, co-facial deposition was more challenging for test sensors designed to detect LE in urine given the need for two isolated reagent formulations that are resuspended sequentially. In the primary embodiment, the ester-containing substrate again was 4-Hydroxyphenyl (N-(3-(methoxycarbonyl)pyridine-5-sulfonyl)-L-alaninate. Each molecule of ester-containing substrate releases one molecule of hydroquinone upon ester hydrolysis, which is detected by the screen-printed electrodes. A urine fluid sample is applied as a droplet and enters the flow cell by active fluidic metering to sequentially resuspend each of the dried reagent formulations in two consecutive deposition zones of the test sensor before advancing to the end of the flow cell to completely cover the working electrode. After a brief incubation to allow for enzyme-specific hydrolysis of the chemical substrate by LE, the activity of the enzyme is detected. Test sensors for detecting LE in urine could also be designed for passive fluidic using a hydrophilic coated top film to form the flow cell.

While test sensors prepared with deposition of glycine buffer onto the hydrophilic flow cell demonstrated poor fill characteristics due to the effect of the buffer on the surface energy within the passive flow cell, the reagent buffer has no impact on fill characteristics of test sensors that either do not rely on passive filling (active pumping or metering) or do not rely on depositing the reagent formulation directly onto the hydrophilic coated surface. In such cases, glycine buffer, pH 3.3 provided superior performance compared to acetate buffer, with improved signal to noise from reduced background.

TABLE 5
Theoretical reagent concentrations after
complete resuspension in urine sample

| | Amount | Units |
|---|---|---|
| Main Reagent Component | | |
| Myoinositol | 0.18% | w/v % |
| CHAPS Hydrate | 0.53 | w/v % |
| Glycine Buffer, pH 3.3 | 5.26 | mM |
| Trehalose | 1.05 | w/v % |
| Triton X-100 | 0.19 | w/v % |
| Igepal CA-630 | 0.24 | w/v % |
| α-Cyclodextrin | 0.68 | w/v % |
| Ester Substrate | 31.6 | mM |
| Pre-Treatment Reagent Component | | |
| Phosphate buffer, pH 7.4 | 250 | mM |
| Sodium Iodate | 80.5 | mM |
| CHAPS | 1.75 | w/v % |
| Myoinositol | 0.70 | w/v % |

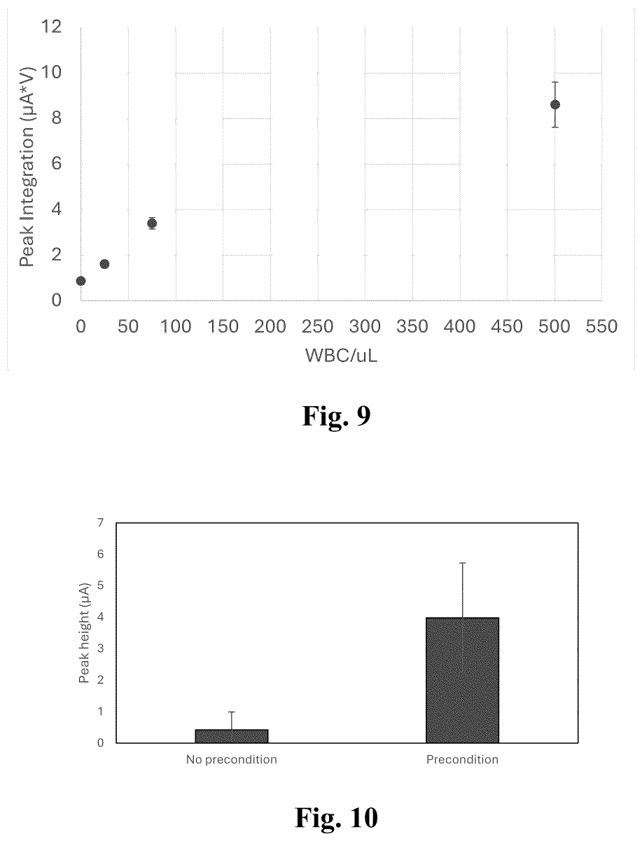

Test sensors were prepared and tested with artificial urine samples, and calibration based on the level of spiked WBCs (FIG. 9). The assay demonstrated a limit of detection of 6.7 WBCs per μL, linear range from 25 to 500 WBCs per μL, and good within-run precision with a coefficient of variation of 7.4%.

With test sensors prepared for the detection of LE in synovial fluid, the detection method was based on integration of oxidation peaks of hydroquinone released from the ester-containing substrate. However, upon spiking hydroquinone into a series of urine samples, there were native oxidation peaks due to the presence of potentially interfering substances that are common in urine. Therefore, detection of LE in urine in the oxidation window was found to be unsuitable. On the contrary, there was a distinct integration peak without obvious interfering species in the reduction window. Detection of the reduction signal of hydroquinone released from the ester-containing substrate was performed with one of two electrochemical techniques: either square wave voltammetry using an overpotential from +0.2V to −0.6V or chronoamperometry at −0.5V for 3 seconds. Upon spiking hydroquinone into a series of 9 urine samples, the reduction peak was found to be less than expected based on the level of hydroquinone in the urine sample. It was hypothesized that this was due to the fact that the hydroquinone mediator was in a partially reduced state in urine. A pre-condition step was implemented to first fully oxidize all of the hydroquinone in the sample by applying a positive voltage from +0.5 to +1.0V immediately prior to performing the electrochemical analysis technique. The pre-condition step completely restored the reduction peak by eliminating the partially reduced hydroquinone mediator (FIG. 10), but also requires a fast scan time for the electrochemical analysis to minimize spontaneous reduction of the mediator after the pre-condition step. Reducing the scan time with square wave voltammetry from 80 seconds to 2 seconds significantly increased the average reduction peak height from 2 μA to 30 μA and reduced bias from 17% to less than 10% in urine samples. A quick 3 second scan with chronoamperometry also demonstrated bias and imprecision each below 10%.

Unlike synovial fluid which has a narrow pH range close to 7.4, the pH of urine typically varies from 4 to 9, and thus urine is not a suitable matrix to use as a buffer to restore neutral pH conditions of the reaction solution. The buffering capacity of phosphate and HEPES at final concentrations from 50 to 250 mM were first evaluated with 6 urine samples. The average deviation from target pH 7.4 was lowest with 250 mM phosphate buffer (Table 6). A 250 mM Phosphate buffer was then tested further with 28 clinical samples, producing an average deviation from target pH 7.4 of 0.05 pH units with a coefficient of variation among urine samples of 0.88%. Test sensors were then prepared with a pre-treatment reagent formulation with 250 mM phosphate buffer deposited and dried onto the pre-treatment deposition zone. Test sensors were evaluated with 7 urine samples spiked with hydroquinone mediator, producing an average peak area of 3.1 μA*V with a coefficient of variation of only 6.5%. The average pH deviation from the target pH with a dried pre-treatment reagent was 0.16 pH units. This demonstrated the capacity for the dry pre-treatment reagent to be rapidly resuspended and restore the neutral pH of the urine sample.

TABLE 6
Deviation of buffered urine samples from target pH

| Buffer | Concentration | pH Deviation |
|---|---|---|
| Phosphate | 50 mM | 0.20 ± 0.15 |
| Phosphate | 100 mM | 0.14 ± 0.09 |
| Phosphate | 250 mM | 0.06 ± 0.03 |
| HEPES | 50 mM | 0.15 ± 0.14 |
| HEPES | 100 mM | 0.08 ± 0.07 |
| HEPES | 250 mM | 0.11 ± 0.05 |

The KIDNEYS produce urine as a filtrate to eliminate waste chemicals from the blood. Therefore, urine commonly contains many potentially interfering substances that make it a challenge for in vitro diagnostics. A range of potential interferents found in urine were tested, including urea, creatinine, oxalate, citrate, and ammonium chloride, with no significant bias (<15%). However, ascorbate, a common interferent frequently found in urine, was found to cause a clinically significant negative bias for the detection of LE in urine of 23% at 250 mg/L and 42% at 500 mg/L. Individuals taking vitamin C supplements typically have urine ascorbate levels ranging from 100 to 500 mg/L, and ascorbate levels above 500 mg/L are less common only in individuals consuming very large doses of vitamin C. Test sensors for the detection of LE in urine were prepared with the addition of sodium iodate to the pre-treatment reagent. The addition of a sufficient amount of iodate to the pre-treatment reagent to neutralize ascorbate resulted in a significant reduction in bias from 23% without iodate to 3% with iodate at 250 mg/L ascorbate and from 42% without iodate to 10% with iodate at 500 mg/L. Chloramine, a water disinfectant similar to chlorine, was also able to neutralize ascorbate and restore recovery of LE, but hydrogen peroxide could not neutralize ascorbate.

TABLE 7
Percent recovery of leukocyte esterase in the presence
of ascorbate with and without iodate neutralization

% Recovery

| Ascorbate (mg/L) | Iodate | No Iodate |
|---|---|---|
| 0 | 100% | 100% |
| 100 | 98% | 96% |
| 250 | 97% | 77% |
| 500 | 90% | 58% |

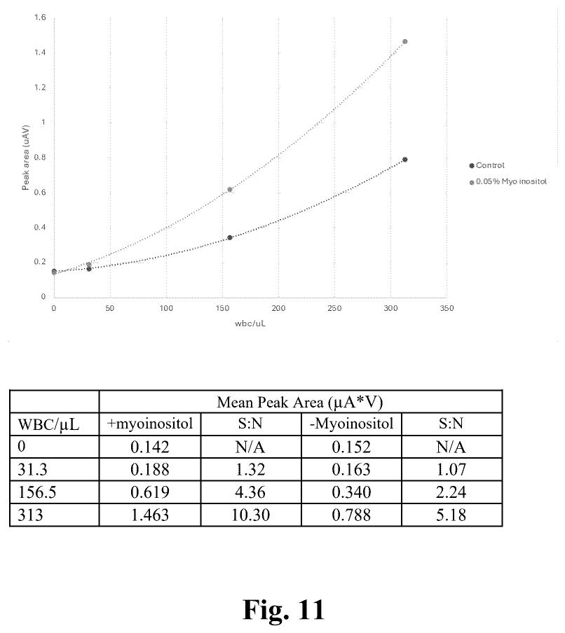

Myoinositol is a sugar that can improve the properties of the dry reagent cake to enhance resuspension and solubilization of dried reagents. Test sensors for the detection of leukocyte esterase in urine were prepared with and without the presence of myoinositol (0.05%) in the dry chemistry reagent formulation. A serial dilution series of artificial urine spiked with WBCs from 0 to 313 WBC/μL was prepared and tested with both sets of test sensors. The addition of myoinositol dramatically improved signal to noise by almost two-fold at 156.6 and 313 WBC/μL by significantly increasing the signal in the presence of WBCs with, surprisingly, no change in background noise (FIG. 11).

These and other advantages maybe realized in accordance with the specific embodiments described as Well as other variations. It is to be understood that the above description is intended to be illustrative, and not restrictive. Many other embodiments and modifications within the spirit and scope of the claims will be apparent to those of skill in the art upon reviewing the above description. The scope of the invention should, therefore, be determined with reference to the appended claims, along with the full scope of equivalents to which such claims are entitled.

What is claimed is:

1-59. (canceled)

60. A biosensor comprising:
a surface;
at least one electrode on said surface;
a reagent on at least a portion of said surface, said reagent comprising:
an ester-containing substrate with an electrochemical mediator;
a buffer having a pH sufficiently low to preserve said substrate prior to introduction of a sample having a higher pH;
one or more chemical stabilizers; and
one or more surfactants.

61. The biosensor of claim 60, wherein said buffer has a pH of less than 4.

62. The biosensor of claim 60, wherein said buffer is one of acetate, glycine, or citrate.

63. The biosensor of claim 60, wherein said buffer is acetate or glycine with a pH of between 2 and 4.

64. The biosensor of claim 60, wherein the final concentration of said ester-containing substrate is about 1 mM to about 50 mM.

65. The biosensor of claim 60, wherein the ester-containing substrate is hydrolyzed by leukocyte esterase (LE).

66. The biosensor of claim 60, wherein the ester-containing substrate is:

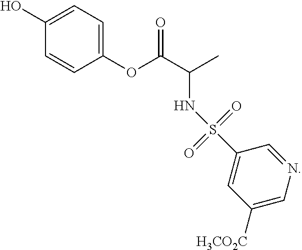 [SMILES: COC(=O)c1cncc(S(=O)(=O)NC(C)C(=O)Oc2ccc(O)cc2)c1]

67. The biosensor of claim 60, wherein at least one of said one or more surfactants is non-ionic, wherein said one or more surfactants is at least 0.1% w/v of said reagent.

68. The biosensor of claim 67, wherein said non-ionic surfactant is at least one of a polyethylene oxide surfactant, a steroidal saponin surfactant, or a zwitterionic surfactant.

69. The biosensor of claim 68, wherein said non-ionic surfactant is one or more of Triton X-100, Triton X-114, or IgepalCA-630, digitonin, or 3-[(3-cholamidopropyl)dimethylammonio]-1-propanesulfonate (“CHAPS”).

70. The biosensor of claim 69, wherein CHAPS is between about 0.01% and 10%, or about 0.1-5%, or about 1-2% w/v.

71. The biosensor of claim 67, wherein non-ionic surfactant is between 0.01% and 1% w/v, or between 0.05% and 0.25% w/v of said reagent.

72. The biosensor of claim 67, wherein said non-ionic surfactant is a combination of said non-ionic surfactants.

73. The biosensor of claim 72, wherein said non-ionic surfactant is a combination of Triton X-100 and Igepal CA-630, each between 0.05% and 0.25% w/v.

74. The biosensor of claim 60, wherein said reagent is not in contact with said electrode.

75. The biosensor of claim 74, further comprising a microfluidic channel defined on said surface, and herein said reagent is dried within said microfluidic channel.

76. The biosensor of claim 75, wherein said reagent is dried upstream from said electrode so as not to be in contact with said working electrode.

77. The biosensor of claim 75, further comprising:
at least one pre-treatment reagent along said microfluidic channel between said sample inlet and said reagent, and comprising a concentrated pH neutralization buffer, an ascorbate neutralizer, and optionally a surfactant either separate or in combination.

78. The biosensor of claim 77, wherein said pre-treatment reagent is dry deposited within said microfluidic channel upstream from said reagent so that said pre-treatment reagent is resuspended into a biological sample before said sample contacts said reagent.

79. A method of using the biosensor of claim 60, wherein said reagent has a first pH, said method comprising:
combining said reagent with a test sample causing said first pH to increase to a second pH.

80. The method of claim 79, wherein test sample is one of synovial fluid, cerebrospinal fluid, or urine.

81. The method of claim 60, wherein prior to combining said test sample with said reagent formation, said test sample is first combined with a pre-treatment reagent comprising a concentrated pH neutralization buffer such that the combination of a buffered test sample and said reagent increases said first pH to a second pH, wherein said second pH is at or near PH of said pH neutralization buffer.

82. The method of claim 60, wherein second pH is at or near pH of said test sample.
